(19) United States
(12) Patent Application Publication
(10) Pub. No.: US 2010/0135980 A1
(43) Pub. Date: Jun. 3, 2010

(54) Therapeutic and prophylactic uses of cell specific carbonic anhydrase enzymes in treating aging, disorders of aging, cancer, as growth factors, and as an alternative to stem cell therapy

(76) Inventor: Victorio C. Rodriguez, Parma, OH (US)

Correspondence Address: VICTORIO C. RODRIGUEZ, 7791 HOERTZ, PARMA, OH, 44134, US

(21) Appl. No.: 12/655,783

(22) Filed: Jan. 7, 2010

Related U.S. Application Data
(63) Continuation-in-part of application No. 11/801,870, filed on May 12, 2007.
Application No. 11/801,870 is a continuation-in-part of application No. 10/858,091, filed on Jun. 1, 2004, now Pat. No. 7,256,184.
Application No. 10/858,091 is a continuation-in-part of application No. 10/077,719, filed on Feb. 15, 2002, now Pat. No. 6,821,997.
Application No. 10/077,719 is a continuation-in-part of application No. 09/688,290, filed on Oct. 16, 2000, now abandoned.

(51) Int. Cl. A61K 38/51; A61P 35/00; A61P 19/08; A61P 19/02; A61P 37/00; A61P 25/00; A61P 17/00

(52) U.S. Cl. 424/94.5

(57) ABSTRACT

A method for the treatment and prophylaxis of diseases or conditions associated with aging which includes oxidative stress and cancer. Such diseases or conditions are associated with a decrease or increase presence of one or a combination of one or more Carbonic Anhydrase isozymes I TO VII in the tissue of a subject. These diseases include but are not limited to chronic neurodegenerative diseases such as Alzheimer's disease, parkinson's disease, multiple sclerosis, autism, lou gehrig's disease, huntington's disease, immune disorders which includes systemic lupus erythematosus, diabetes mellitus; musculo-skeletal disorders which includes osteoporosis,arthritis, osteomalacia. Cancer which includes the breast, lung, colon, prostate gland, ovary, esophagus, liver, rectum. The method comprises administering to the patient a pharmaceutically effective non toxic amount of one or a combination of one or more compounds that increases or decreases the level of one or more Cell Specific Carbonic Anhydrase Enzymes whose levels have been increased or decreased in the tissue of the subject. Such compound maybe the Cell Specific Carbonic Anhydrase Enzymes I to VII. A compound that when absorbed reacts or dissociates to form cell specific carbonic enzymes or one or more compounds that when administered promotes the increase or decrease level of one or more cell specific Carbonic anhydrase I,II,III,IV,V,VI,VII. As growth factors of stem cells as an alternative to stem cell therapy. These methods includes the administering of one or a combination of one or more of these compounds over an extended period of time ranging from 6 months until the patient dies.

### CROSS REFERENCE TO RELATED APPLICATIONS

This application is a continuation in part of co-pending U.S. patent application Ser. No. 11/801,870 filed on May 12, 2007 which is a continuation-in-part of U.S. application Ser. No. 10/858,091 FILED ON June 1, now U.S. Pat. No. 7,256,184,WHICH IS A CONTINUATION IN PART OF U.S. application Ser. No.10/077,719 FILED ON Feb. 15, 2002 now U.S. Pat. No. 6,821,997 WHICH IS A CONTINUATION IN PART OF U.S. application Ser. No. 09/688,290 FILED ON Oct. 15, 2000 NOW ABANDONED.

### FIELD OF INVENTION

This invention deals with the therapeutic and prophylactic uses of cell specific carbonic anhydrase enzymes I,II,III,IV,V,VI, VII; activators and inhibitors. Treating diseases or conditions associated with an increase or decrease in the level of one or a combination of one or more cell specific carbonic anhydrase enzymes I to VII in the tissue of the subject. This includes aging; disorders of aging including oxidative stress and cancer. As growth factors of stem cells and as alternative to stem cell therapy.

### BACKGROUND OF INVENTION

Carbonic Anhydrase Enzymes are ancient Zinc enzymes which are further divided into alpha, beta and gamma carbonic anhydrase enzymes as illustrated in FIG. 4. The ALPHA GROUP belongs to mammals and are thought to be 200 TO 300 million years old. The BETA GROUP-belongs to plants are about 3.5 billion years old. The Gamma group belongs to the methane producing bacteria which are also about 4 billion years old. These Carbonic Anhydrase Enzymes are structurally distinct from each other except they contain the same Zinc ion and catalyzing the same reversible reaction H₂O+CO₂—H++HCO₃₋

Carbonic anhydrase enzymes are the fastest enzymes doing 10(6) power reactions per second. In mammals, their activities are not limited to maintaining the acid base balance, maintaining the PO₂/CO₂ ratio, ester hydrolysis, ion transport, phospatase activity, CO₂ hydration, maintaining the equilibrium between H₂O, CO₂ and H+HC0₃₋ in all spaces, cellular, interstitial and vascular spaces as illustrated in FIG. 3.

In Mammals Carbonic Anhydrase Isozymes (Genes) are further divided into: Cell Specific Carbonic Anhydrase I to XIV:

CA-I are cytosolic enzymes are mostly found in Red blood Cells, vascular endothelial cells, gastrointestinal mucosa

CA-II are cystosolic enzyme found in almost all tissues but mainly in the Brain, Bones, Kidneys

CA-III—cystosolic enzyme mostly found in muscles, synovial tissues

CA-IV is membrane bound, found in the capillary endothelial cells, brain and kidney

CA-V mitochondrial-beta cells, pancreas

CA-VI secretory found in tears saliva, milk, mammary gland, nasal and respiratory mucosa

CA-VII cystosolic Enzyme-hippocampal CA I pyramidal cells, salivary Glands

CA VIII—CARP(carbonic anhydrase related protein)

CA-IX—Membrane Bound MN/protein(tumor associated)—Catalytic

CA-X—CARP(carbonic anhydrase related proteins)

CA-XI—CARP(carbonic anhydrase related proteins)

CA-XII—Membrane Bound

CA-XIII—cystosolic

CA-XIV—Membrane Bound

Normal aging in humans is recognized as producing some or all of the following typical physiological results but not limited to:

1. Brain weight is reduced by 15%

2. Blood flow to the brain is reduced by 20%

3. Body water content is reduced by 18%

4. Body weight is reduced by 12%

5. Nerve conduction velocity is reduced by 10%

6. Number of nerve fibers are reduced by 37%

7. Decreased amounts of enzymes and coenzymes

8. Decreased amounts of neurotransmitters

9. Depletion of phosphorelative enzymes

10.Apostosis-chronic neuronal atrophy

Signs and Symptoms of Human Aging includes but are not limited to:

1. increased body fat

2. decreased Energy

3. Decreased Sexual Performance

4. Decreased Libido

5. Decreased Skin elasticity

6. Decreased Mental Function

7. Decline in Vision/eyesight

8. Decreased Strength

9. Joint Pain

10. Decreased Lean Muscle

11. Decreased Bone Mass

12. Decline in Memory

13. Diminished Immune System

14. Wrinkles and Cellulite

15. Decreased Cardiac Output

16. High Blood Pressure

17. Undesirable Cholesterol

18. Decreased Exercise Performance

19. Slower rate in Healing

20. Decline in taste/smell

21. frailities and others

In describing their work in an article entitled “Studies on age-Dependent Ozonide Changes in Human Cerebral Cortex,” (by Reichlmeier K., Ermini M., and Schlecth H. P.—Aktuellel Gerontl 1978 Aug. 8(8):44-8) the authors report that they investigated the activity of various enzymes of human brains obtained at autopsy and covering an age range of 19 to 91 years. Protein Kinase, which mediates the information carried by the second messenger, cyclic AMP(3′,5′-cyclic adenosine monophosphate), does not show age-related changes of basal activity. Cyclic AMP-dependent activation of protein kinase remains constant up to 60 years of life but it undergoes a distinct and progressive decline between 60 and 90 years. In the corpus striatum, no age related changes of cyclic AMP-dependent protein activity were observed. The activity of carbonic anhydrase exhibits, in both human cortex and corpus striatum, an age-dependent decrease that also begins after the sixth decade.

Aging is further divided into primary aging and secondary aging (accelerated). Primary aging is caused mainly in the inherent progressive decrease activities of the genes that regulates the cell. While secondary aging (accelerated) is caused mainly by oxidative stress. Oxidative stress is caused mainly by external factors such as environmental factors, toxic materials, ionizing radiations, or any disease or condition that alters the vascular endothelium and the cell wall membrane. These altered conditions allows the entry of substances that will displace the zinc from cell specific carbonic anhydrase enzymes leading to the decreased level of cell specific carbonic anhydrase enzymes hence leading to cellular death.

In Aging there is an age-associated dysregulated inflammatory and Immune Response. There is an increase production of PGE2 and Cyclooxygenase which inhibits the production of Cell Specific Carbonic Anhydrase Enzymes

IN Brain Behav Immun. 2004 November; 18(6):487-94 by Wu D, Meydani S N Mechanism of age-associated up-regulation in macrophage PGE2 synthesis Many physiological functions of the body change during the aging process. Disregulated immune and inflammatory responses have been well documented in both humans and animals. The investigation into the cellular and molecular mechanism underlying these disorders has provided compelling evidence that up-regulated cyclo-oxygenase (COX2) and its product particularly prostaglandin (PGE2) lay a critical role in the age-associated disregulation of the immune and inflammatory responses. Studies have shown that increase production of PGE2 by old macrophages contributes to the suppresion of T cell function. Decreasing production of PGE2 have shown enhanced T mediated cell function. Upon Stimulation, old mice macrophages generate more ceramide which in turn augments stimulated COX2 expressions and PGE2 production.

Crit Rev Immunol. 2004; 24(5):349-62 by Trottein F, Faveeuw C, Gosset, P, Angeli V

### Role of the D Prostanoid Receptor 1 in Modulation of Immune and Inflammatory Responses.

Prostaglndins (PGS) are potent eicosanoid lipid mediators derived from phospholipase released arachidonic acid, which are involved in numerous homeostatic biological functions and inflammation. Along with their role in inflammatory responses evidence strongly suggest that PGS, including PGD2 are part of the complex regulatory network that modulates the immune system. PGE2 is the major prostaglndin activated by mast cells in allergic diseases.

In Arthritis Res. 1999; 1(1):63-70. Epub 1999 Oct. 14. by Tetlow L C, Wooley D E

The effects of alpha, 25-dihydrovitamin D3 on matrix metalloproteinase and prostaglandin E(2) production by cells of rheumatoid lesion. Vitamin D3 modulate the effect of Matrix metalloprotienase. Matrix metallo proteinase and PGE2 are chondrolyic enzymes which plays a major role in the breakdown in the rheumatoid joint.

Arthritis Rheum. 1999 December; 42(12):2561-8 by Yaron I, Shirazi I, Judovich R, Levartovsky D, Caspi D, Yaron D

Fluoxetine and aminitriptylline inhibit nitric oxide, Prostaglandin PGE2 and hyaluronic acid production in human synovial cells and synovial tissue cultures. Their conclusion; Inhibition of NO and PGE2 production by connective tissue cells is a mechanism by which some antidepressant medications may affect pain, articular inflammation.

In cystic fibrosis

Eur J cell Biol; 2002 August 81(8):437-47

Targeting of carbonic anhydrase IV to plasma membranes is altered in cultured human pancreatic duct cells expressing a mutated (deltaF508) CFTR-by Fangul M., Salvador C, alvares L., Cantet S., Hollande E

Their studies that the level of Carbonic anhydrase IV is decreased resulting in the decreased secretion of CL− and HCO3− ions as well as defective targeting of other proteins. This is due to a 6-10 fold fewer cells in the CFPAC-1 cell lines.

ANTIBODIES to Cell Specific Carbonic Anhydrase Enzymes are produced as a result of an immunologic response to exposure to antigens in Humans. These antigens try to destroy these enzymes and so the human body try to defend itself by producing cell specific carbonic anhydrase antibodies.

Antibodies to Carbonic anhydrase has been found in systemic lupus erythematosus and other rheumatic diseases; Arthritis Rheum. 1992 January; 35(1):73-82 by Itoh Y, Reichlin M. They found the increase of autoantibodies of CA I, CA II isoform, in Systemic Lupus erythematosus, scleroderma, and polymyositis in their sera.

In Dermatol Sci. 1991 May; 2(3):147-54 by Inagaki Y, Jinno-Yoshida Y, Bamasaki Y, Ueki H. they also found auto Antibodies to CA I, CA II isoform in patients sera in Sjogren's Disease, Renal tubular acidosis and systemic Lupus Erythematosus.

Autoimmunity. 2003 March; 36(2):85-9 by Alessandri C, Bombardieri M, Scrivo R, Viganego F Conti F, deLuca N, Riccieri V, Valeseni G deals with the presence of anti-Carbonic anhydrase antibody as possible pathogenic role of anti-CA II in the development of lung damage in systemic sclerosis (SSC) disease.

In Clinical Chemistry. 2003; 49:1221-1223—Effect of Anti-Carbonic anhydrase antibodies on Carbonic anhydrase I and II by Francesco Botre, Claudio Botre, Elisabetta Podesta, Mauro Podda and Pietro Invernizzi. they isolated CA I,II isoform antibodies in patients with systemic lupus erythematosus, polymyositis, systemic sclerosis, endometriosis syogrens, disease, idiopathic chronic pancreatitis, primary biliary cirrhosis, and autoimmune cholangitis.

Int J Mol Med. 2002 May; 9(5):499-502 by Andoh A, Fujiyama Y, Yoshioka U, Sasaki M, Araki Y Tsugikawa T—discusses the presence of Anti-carbonic anhydrase II antibodies in patient with ulcerative colitis.

Growth Factor are useful in the enhancement maintenance and differentiation of stem cells to specific cells or tissues.

In Proc Soc Exp Biol Med. 1987 January; 184(1):24-30 by Cavral A T R, Hewett-Emmet DWelty R J, Castor C W Effects of human Carbonic anhydrase III on synovial and muscle fibroblastic glycosaminoglycan metabolism

They investigated the ability of CA III, isolated from adult human skeletal muscle to regulate cell growth and glycosaminoglycan formation in connective tissue cells. Cell culture experiments showed that exposure to CA III by synovial connective tissue fibroblast, they were increased by 2 to 12 fold, also increased hyaluronic acid synthesis. Also exposure to CA III leads to an increase in muscle fibroblast by 20-45%, synovial fibroblasts by 16-70%.

Endocrinology. 1997 November; 138(11):4852-7. by Biskobing D M, Fan D, Fan X, Rubin J. Induction of carbonic anhydrase II expression in osteoclast progenitors requires physical contact with stromal cells.

It tells that osteoclast progenitors requires the physical communication of Carbonic anhydrase II with stromal cells for them to differentiate to osteoclast.

J. Histochem Cytochem. 2004 August; 52(8):1057-62 by Kimoto M, Iwai S, Maeda T, Yura Y, Fernley R T, Ogawa Y

Tells us that CA VI plays A role in olfactory function as a Growth factor in the maturation of the olfactory epithelial cells.

Am J Med Sci. 1999 December; 318(6):39-405—by Henkin R I, Martin B M, Agarwal R P

Efficacy of Exogenous Oral Zinc in Treatment of Patients with Carbonic Anhydrase VI Deficiency.

Tells us about the use of oral Zinc in stimulating the production of CA VI which promotes the growth of taste buds.

In Transplantation. 2001 Jun. 27; 71(12):1735-40 by Nakano K, Migita M, Mochisuki H Shimada

T Bone Marrow Transplantation was reported effective in preventing the progression of neurological deterioration of lysosomal disease. They concluded that the bone marrow contains cells capable of differentiating into oligodendrocytes, astrocytes and microglia when exposed to brain microinvironment.

J Histochem Cytochem. 2004 August; 52(8:1107-12 by Leinonem J S, Saari K A, Seppanem J M, Myllyla H M, Rajaniemi their studies shows that CA VI maybe a growth factor in the respiratory epithelium

Brain Res Dev Brain Res. 1992 Jun. 19; 67(2):257-63 by Cammer W, Zhang H shows that Carbonic anhydrase are distinct precursors of astrocytes and oligodendrocytes in the forebrains of neonatal and young rats.

The American Society for Biochemistry and Molecular Biology, Inc. Volume 271, Number 17, Issue of Apr. 26, 1996 pp. 10-169-10174 Peter H. Frederikse, Donita Garland, J. Samuel Sigler Jr., Goran Piatigorsky In their studies, oxidative stress increases the production of B amyloid precursor proteins and B amyloids (AB) in mammalian lenses. Amyloid diseases are characterized by protein aggregations linked to Oxidative stress.

Rinsho Byori. 2003 February; 51(12):140-5 Amyloidosis and Oxidative stress—Nakamura M, Ando Y.

Their studies on oxidative stress have revealed that free radical injury appears to be involved in either the amyloid formation process or in the post-fibrillar modification in several types of amyloidosis the role of oxidative stress in the pathogenesis of dialysis related amyloidosis and familial amyloidotic polyneuropathy.

Journal of Neuro-inflammation 2004, 1:21 doi:10.1186/1742-2094-1-21 Yuemang Yu, Cinzia Cinici, Hanguan Tang, John Q Trajanowski, Virginia My Lee, Domenico Pratico Brain Inflammation and oxidative stress in a transgenic mouse model Of Alzheimer like brain amyloidosis. Their studies implicates an increasing body of evidence which implicates both brain inflammation and oxidative stress in the pathogenesis of alzheimer's disease.

In autism there are evidences the Mercury (Hg), a neurotoxic metal causes autism. Mercury displaces the Zinc ion found in Cell specific Carbonic anhydrase enzymes resulting in their decreased level leading to cellular death.

In U.S. Pat. No. 6,297,212 and U.S. application Ser. No. 09/933,039—Gregory Fahy, he used growth hormone and dehydroepiandrosterone(DHEA) in regenerating the involutedly thymus in restoring the immune system.

Below are selected patent references, other patent references published on diseases of oxidative stress are incorporated in this invention.

1. U.S. Pat. No. 6,306,844—use of alpha methyl-19 nor 20(s) 1,25ldihydroxyvitamin D3 to increase bone strength

2. U.S. Pat. No. 6,150,346—Method and composition for treating Or preventing osteoporosis.

3. U.S. Pat. No. 6,930,099—composition for the treatment and prevention of endothelial dysfunction

4. U.S. Pat. No. 5,700,790—prevention and treatment of myocardial failure

5. U.S. Pat. No. 6,297,212—Growth Hormone therapy and related methods

6. U.S. Pat. No. 5,763,428—Method of treating skin disorders with Vitamin D4

7. U.S. Pat. No. 5,763,429—Method for treating prostatic diseases using Vitamin D analogues

8. U.S. Pat. No. 5,895,652—metabolic adjuvanation and cellular repair

9. U.S. Pat. No. 7,078,059—treatment of bone diseases

10. U.S. Pat. No. 6,828,331—growth hormone secretagogues

### Cancer

Cancer is the uncontrollable growth of cells originating from normal tissues and the property of killing the host by means of local extension or distant spread. They are unresponsive to normal regulatory factors, they lose their property to differentiate and lose the function of normal tissue of origin.

Cancer is further divided into undifferentiated forms and well differentiated forms. Undifferentiated forms are immature and do not look like the tissue it arose and very aggressive while the opposite is true for the differentiated form.

Cell specific carbonic anhydrase enzymes are growth factors of stem cells. Abnormal expressions of Cell specific carbonic anhydrase enzymes maybe due to Primary Gene mutation (Hereditary) and Secondary Gene mutation (due to oxidative stress). These mutant Genes causes abnormal growth of cells in the form of new-growths such as cancer. These mutant genes could produce either an increased or decreased levels of cell specific carbonic anhydrase enzymes. The decreased levels of Cell Specific Carbonic Anhydrase Enzymes, results to: the cells are unable to differentiate to mature cells such as found in undifferentiated forms of cancers. Excessive production of cell specific carbonic anhydrase enzymes causes excessive production of cellular elements such as found in well differentiated forms of cancer.

In Experimental Oncology 22, 162-164, 2000(September) by I. Puscas, C. Puscas, M. Coltau, M. Stamm, G. Domuta

The serum of Carcinoma patients powerfully activates Carbonic Anhydrase II in cancer of the Esophagus, Colon and rectum, liver, Pancreas, lung, breast, ovary, testis, prostate while the sera of non cancer patients and normal patients are devoid of such activity. They observed that the activatory effects of cancer serum upon CA II do not seem to correlate with the localization, staging or histological type of tumor.

CA IX—Specific inhibitors-by Supuran Claudiu et al U.S. application Ser. No. 10/723,795 published on Jul. 29, 2004—, now U.S. Pat. No. 7,550,424 published on Jun. 23, 2009 tells us about the presence of Carbonic anhydrase isozyme CA IX(MN) oncogene protein. The use of Carbonic Anhydrase IX specific inhibitors.

Eur J Med Chem 2000 September; 35(9):867-74 by Supuran C T, Scozzafava A. teaches the use of Sulfonamide inhibitors as efficient inhibitor 1,3,4-thiadozole-2-sulfonamide against CA II, CA VI of tumor cell growth—leukemia, non-small cell lung cancer, melanoma, colon, CNS, renal, prostate and breast cell lines. They may also act against CA IX, CA XII, CA XIV cell lines

Rev Med Chem. 2004 August; 4(6):625-32 Cox-2 inhibitors, carbonic anhydrase inhibition and anticancer properties of sulfonamides belonging to this class of pharmaceuticalagents-deals with celecoxib and valdecoxib which have sulfamoyl moties but not refocoxib

In U.S. patent application Ser. No. 10/213,793—filed on Aug. 7, 2002 Abandoned Jul. 21, 2004 Carbonic anhydrase inhibitor—inventor Jaime Masferrer, Baldwin Mo. tells us about the use of carbonic anhydrase inhibitor for the treatment of neoplasia which is a continuation in part of U.S. application Ser. No. 10/366,739 filed on Feb. 14, 2003, abandoned Jan. 27, 2006—Composition of a cyclo-oxyginase inhibitor and a carbonic anhydrase inhibitor for the treatment of neoplasia.

Acetazolamide, a carbonic anhydrase inhibitor was used with several anticancer drug in 1993 as an acidic milieu to make them more cytotoxic to the cells. Anticancer Res. 1993 September-October; 13(5A):1549-56 by Teicher B A, Liu S D, Liu J Y, Holden S A, Herman T S

The above articles do not suggest the assaying for the increased or decreased level of cell specific carbonic anhydrase enzymes CA-I to CA-VII in the tissue the human patient in need for the treatment of neoplasia. Their teachings, suggestions, motivations are different from this invention.

Soduim azide and Potassium Cyanide causes instant death because they inhibit the cytochrome system(ATP production). They bind with cell specific carbonic anhydrase enzymes in the mitochondria of all living things, hence they could be used as anti cancer treatment in minute non toxic and pharmacologic effective doses.

### SUMMARY OF THE INVENTION

THE present invention provides the therapeutic and prophylactic uses of cell specific carbonic anhydrase I to VII, its activators and inhibitors for the treatment of aging and disorders of aging which includes oxidative stress; cancer;as growth factors of stem cells and as alternative to stem cell therapy. These conditions or diseases are associated with either an increased or decreased levels of cell specific carbonic anhydrase enzymes such as found in certain forms of cancers: breast, colon, prostate, esophagus, liver, pancreas, rectum, testis, ovary.

In aging, the decreased levels of cell specific carbonic anhydrase enzymes leads to the decreased levels of Hydrogen ions used as fuel of the ion pump that maintains the integrity of the cell wall membrane, decreased levels of hydrogen ions needed to neutralize all reactive substances leading to oxidative damages to almost all molecules of the subject; and also decreased fuel for other cellular functions. These diseases or conditions include but not limited to immune diseases which includes systemic lupus erythematosus, diabetes mellitus, chronic neurodegenerative disorders which includes but not limited to alzheimer's disease, multiple sclerosis, Lou gehrig's disease, parkinson's disease, autism; bone disorders which includes but not limited to osteoporosis, osteomalacia, osteopetrosis, rickets, arthritis, osteoarthritis, rheumatoid arthritis; amyloidosis; arterioslclerosis; atherosclerosis, cardiomyopathies, endothelial dysfunctions; cystic fibrosis; integumentary dysfunctions which includes wrinkling of the skin; myositic dysfunction; lipid dysfunctions; ischemic heart disease; and other diseases associated with human aging including oxidative stress.

The method comprises assaying for the presence and level of one or a combination of one or more cell specific carbonic anhydrase enzymes in the tissue of a subject and then administering one or a combination of one or more compounds that increases or decreases the level of one or a combination of one or more cell specific carbonic anhydrase enzymes that are present at reduced or increased levels in the tissue of the subject. In certain embodiments the tissue is blood, cerebro-spinal fluid, or the biopsied tissue of the subject. A finding that the tissue level of one or a combination of one or more carbonic anhydrase enzymes is increased or decreased is based upon the comparison with a control value derived from healthy young subjects. In certain embodiments the method comprises administering one or a combination of one or more cell specific Carbonic Anhydrase enzymes I to VII either synthetically produced or naturally produced, to the subject. In certain embodiments the method comprises administering one or more compounds that decreases the level of one or a combination of one or more cell specific carbonic anhydrase enzymes I to VII in the tissue of the subject. In another embodiment comprises administering one or a combination of one or more compounds that induce or promotes the generation of one or a combination of one or more cell specific carbonic anhydrase enzymes I to VII that are present at reduced levels in the subject. In another embodiment the method comprises administering one or a combination of one or more compounds that when absorbed by a tissue of a subject reacts or dissociates to form one or a combination of one or more carbonic anhydrase isozymes that are present at reduced levels in the subject. In another embodiment the method comprises administering one or a combination of one or more cell specific carbonic anhydrase enzymes I to VII as growth factors in replacing damaged or diseased cells or tissues. In another embodiment the method comprises administering Carbonic anhydrase isozymes as an alternative to stem cell therapy in the treatment of damaged, injured or diseased cells or tissues. In another embodiment, administering one or a combination of one or more compounds that induce or promotes the natural generation of one or a combination of one or more Cell Specific Carbonic Anhydrase and VII as growth factors of Stem cells in treating damaged or diseased cells or tissues. In another embodiment, one or a combination of one or more Cell specific carbonic anhydrase enzymes VII are used as antioxidants in treating diseases or conditions associated with oxidative stress. In certain embodiments these one or a combination of one or more compounds are administered over an extended period of time ranging from 6 months till the subject dies.

### BRIEF DISCRIPTIONS OF THE DRAWINGS

FIG. 1. Illustrates Carbonic Anhydrase Enzymes

FIG. 2. Illustrates the pathophysiology of Carbonic anhydrase Isozymes in human diseases

FIG. 3. Illustrates the equilibrium of cell specific carbonic anhydrase enzymes in all spaces in humans

FIG. 4. illustrates the association between the alpha, Beta, Gamma Carbonic anhydrase Enzymes

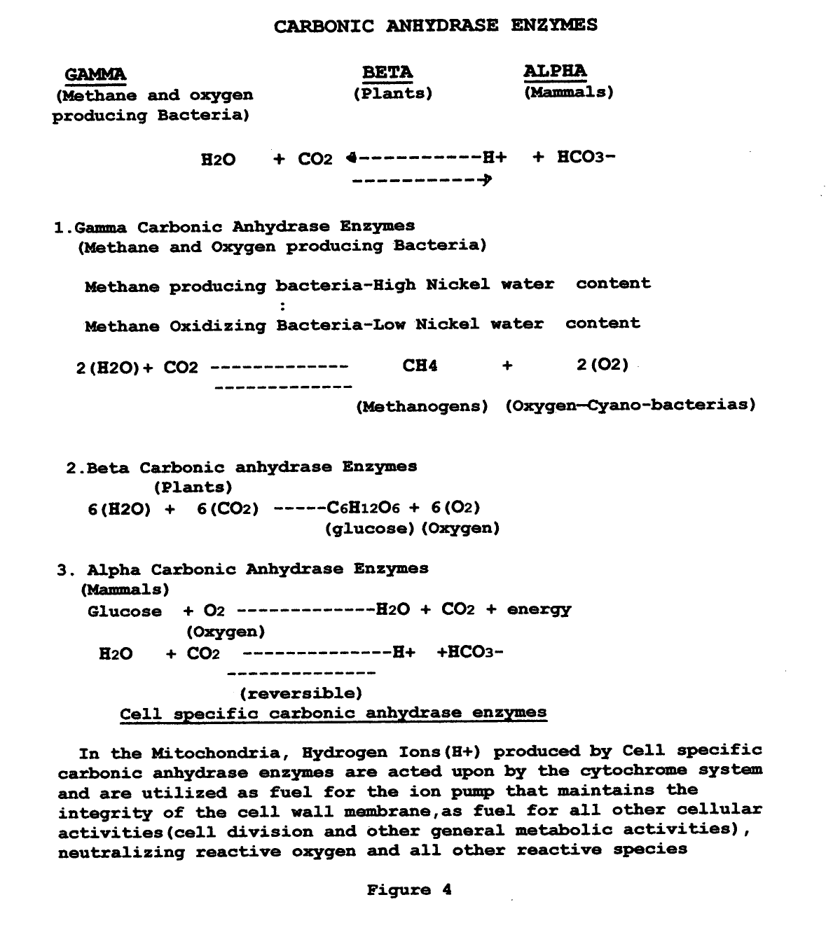

### DETAILED DISCRIPTION OF THE INVENTION

The present invention will now be described by reference to more detailed embodiments, with occasional reference to the accompanying drawling. The invention may, however, be embodied in different forms and shoud not construed as limited to the embodiments set forth herein. Rather, these embodiments are provided so that the disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications patent applications, patents, and other references mentioned herein are incorporated by reference in their entirety.

The following represents an essential chemical reaction that takes place in a mammalian tissue. GLucose (Within the Cells)+Oxygen—H2O+CO₂—H++HCO3− Carbonic Anhydrase present (reversible reaction) Eq. (1)

Glucose is irreversibly oxidized within the cells to produce water and carbon dioxide. In the presence of a catalyst, especially a carbonic anhydrase enzyme (of which several forms exist, of which the form present depends upon the type of tissue cell present), the water and carbon dioxide reversibly produce a hydrogen ion and a bicarbonate ion.

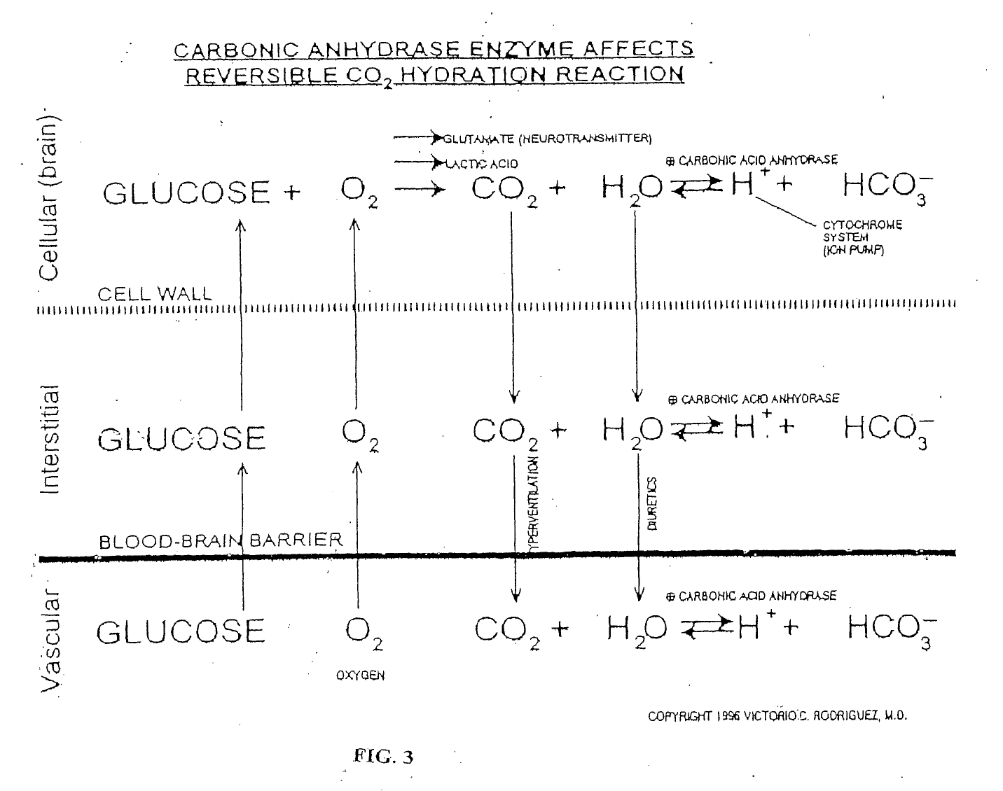

Carbonic Anhydrase is a Zinc-containing enzyme that catalyses the reversible CO.sub.2 hydration reaction illustrated in Eq.1. The mitochondria of cells of different tissues and organs produce different specific carbonic anhydrase enzymes that maintain the equilibrium of the above equation in all spaces-cellular, interstitial, and vascular—as illustrated in FIG. 3. At least seven carbonic anhydrase variants called “S” have been identified. The literature may refer to these as “carbonic anhydrase I through VII) or “CAS-I-VII”.WE refer to this selectivity as “cell specific” and the particular carbonic anhydrase present as being a “Cell specific carbonic anhydrase enzyme”.

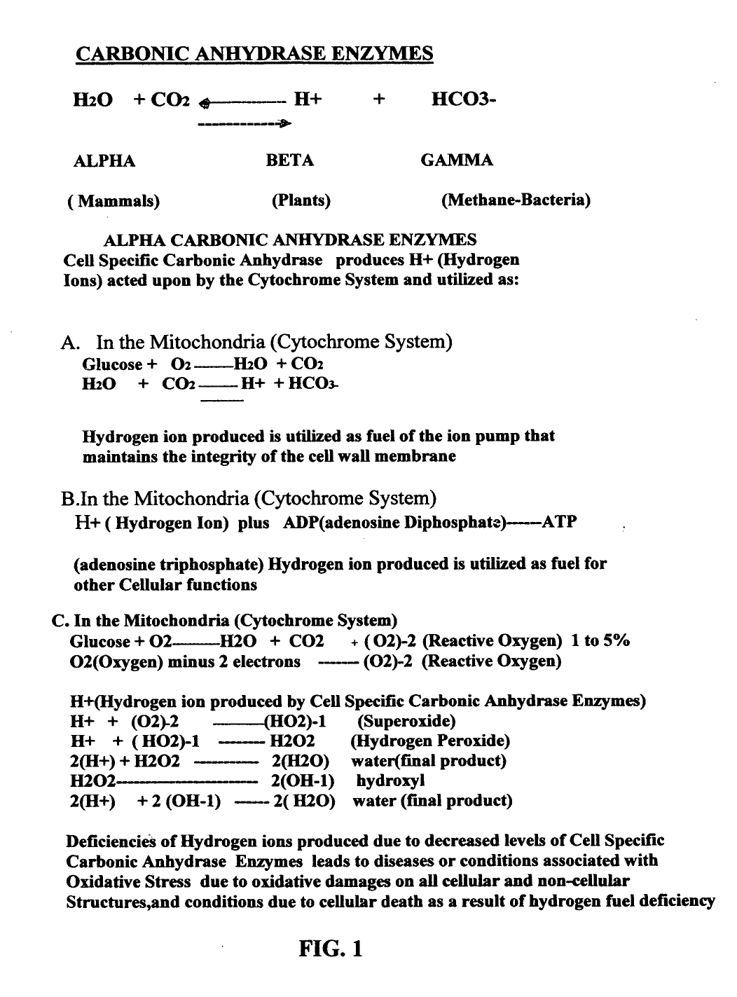

Hydrogen ion produced by carbonic anhydrase enzymes is acted upon by the cytochrome system, which is then utilized as the energy source of the ion pump that maintains the integrity of the cell membrane comprising and enclosing each cell. It is also thought to be the source of the brain's electric current. The proces is schematically illustrated in FIG. 1, presented here with no further discussion.

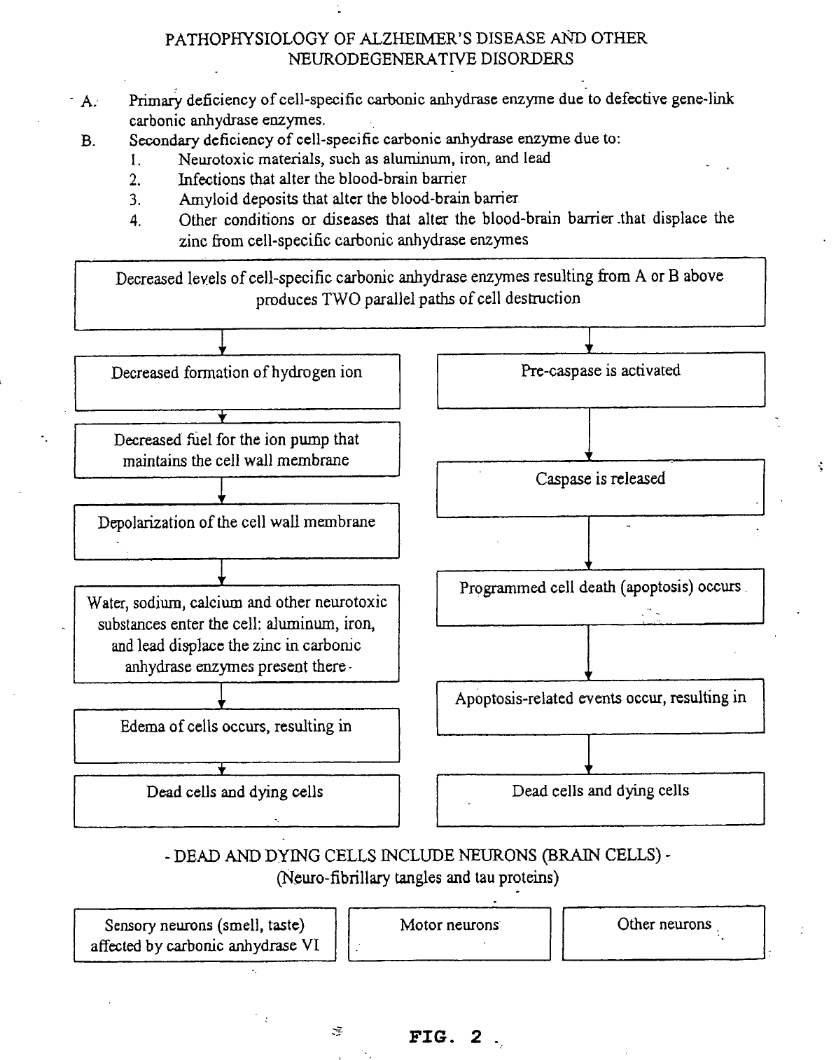

Disruption of the process that include Eq. 1 causes depolarization of the cell wall membrane, hence (Na), water (H.sub.2O), and other chemicals can enter the cell in uncontrollable amounts and potassium (K) can exit uncontrollably, leading to the death and destruction of the involved cells; cellular edema follows. As the edema progresses, the cell dies. Along with the progressive and gradual death of cells, gliosis follows—hence aging in the brain occurs (FIG. 2).

In this newly presented instant invention as illustrated in Fig. I Hydrogen ions produced by cell specific carbonic anhydrase enzymes are acted upon by the cytochrome system and they are utilized as fuel H++ADP—(adenosine diphospate)—ATP(adenosine triphosphate) for other cellular functions (cell metabolism).

Cell Metabolism—ATP(Adenosine Triphospate)—H+(hydrogen)+ADP (Adenosine diphospahte)+energy (½ for cell division and ½ for general cell metabolism)

In this newly presented instant invention as illustrated in FIG. 1 in aerobic respiration, Oxygen is needed in utilizing glucose as the source of energy. As a by product, Oxygen free radicals are produced.

In the Mitochondria 1% to 5% of Oxygen free radicals are formed.

Glucose+O2—H2O+CO2+(O2)−2(reactive Oxygen)

Oxygen−2 electrons—(O2)−2

In the Cytochrome System, Hydrogen ions produced by Carbonic Anhydrase Isozymes also acts as powerful anti oxidant, combining with reactive oxygen as illustrated in the following steps:

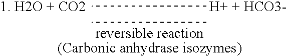

Hydrogen ions produced:(from Carbonic Anhydrase isozymes)

2. H++(O2)−2—(HO2)−1(superoxide)

3. H++(HO2)−1—H2O2(Hydrogen peroxide)

4. H2O2—2(OH−1)(Hydroxyl)

5. 2H++2(OH−1)—2(H2O)(water)-final product

Hydrogen ions produced by carbonic anhydrase enzymes neutralizes all reactive subastances.

Lack of Hydrogen ions due to deficiency of Cell specific carbonic anhydrase enzymes do not complete the above reactions, the final product being water (H2O).This leads to the increase levels of reactive oxygen.

These reactive oxygen causes lipid oxidation, protein oxidation, DNA fragmentations, disruption of the cell wall membrane & binds with almost all molecules in the body. These reactive oxygen species are implicated with diseases associated with oxidative stress which includes but not limited to cancer, aging, immune diseases, atherosclerosis neurodegenerative conditions or diseases.

Hydrogen ions produced by cell specific carbonic anhydrase enzymes are acted upon by the cytochrome system are used by proteins to make them more stable. It has been shown that removing the hydrogen bond leads to protein misfolding leading to diseases and abnormal conditions in a subject which includes amyloidisis.

J Theor Biol. 2002 Apr. 21; 215(4):399-404 Hydrogen Bonds and the Catalytic mechanism of human carbonic anhydrase II-by Thomas S

Tells us the production of hydrogen bonds by carbonic anhydrase II making a substance more stable. Oxidative stress is associated with an increase of amyloid precursor proteins leading to amyloid depositions all over the body of a subject.

### Prior Art

Carbonic anhydrase enzyme has been used to augment the extracellular ph buffering in the cerebral cortex of rats (Journal of Neurophysiology 1995 October 74(4):1806-9. It is known that the blood brain barrier in animals is incomplete compared to that of humans where the blood brain barier is complete and a formidable barrier to chemical transport. Substances that prove efficacious in affecting the brain chemistry of animals are not necessarily efficacious in the brains of human beings because they cannot pass through the more complete blood brain barier in humans. Although some researchers equivocate on this concept most of the medical community accepts the idea that carbonic anhydrase enzymes traverse the blood brain barrier in humans as fact, especially regarding the carbonic anhydrase enzyme referred to as CA-II.

As far as can be determined in the literature, cell specific carbonic anhydrase enzymes have never been used to restore to a higher level the carbonic anhydrase enzymes that are lacking due to decreased levels due to normal aging. At least some of the carbonic anhydrases have been extracted from animal tissue, isolated, and studied for molecular structure. This shows that the enzymes can be isolated and made available for administration to a patient for therapeutic or prophylactic treatment.

In U.S. Pat. No. 5,972,684, Bandman et al. tell us:

Eight enzymatic and evolutionary related forms of carbonic anydrase enzymes are currently known to exist in humans; three cystosolics (CA I, CA II, CA III), two membrane bound forms (CA IV, and CA VII), a mitochondrial form (CA V), a secreted salivary form (CA VI) and a yet uncharacterized Isoforms show a characteristic motif.(See, e.g., http://expassy.hcuge.ch).Though the isoenzymes CA I, CA II, and bovine CA III have a similar secondary structure and polypeptide chain fold, CA I has 6 tryptophans, CA II has 7 and CA III has 8(Boren, K et al. (1996) Protein Sc. 5(12):2479-2484). CA II is the prodominant CA isoenzyme in the brain of mammals.

Inhibition and activation of CA provide information about CA structure and activity. vasodilating prostaglandins E1, E2, E12 inhibit CA in vitro and in vivo and may inhibit the involvement of CA in gastric acid secretion. Nonsteroidal anti-inflammatory drugs which reduce the activity of cyclooxygenase and prostaglandin production have also been observed to activate CA I, and CA II in dose dependent non competetive manner. The pre-prostaglandin cyclooxygenase appears to maintain an inverse relantionship with CA, probably mediated by the ph variations associated with carbonic anhydrase activity

(Puscas, I (1996) J. Pharmacol. Exp. Ther. 277(3):1464-1466). Both prostaglandins E2 and E12 inhibit gastric acid output. Prostaglandin E2 inhibits egress of norepinephrine from sympathetic nerve terminals.

The Bandman et al. teaches another carbonic anhydrase enzyme, CA-VIII, the subject of their patent. The present patent does not deal with nor address CA-VIII

Patients having a carbonic anhydrase VI(CA-VI) deficiency have been treated with orally administered Zinc in an effort to stimulate the synthesis/secretion of CA-VI and the successful results were reported in the American Journal of Medical Science (Efficacy of Exogenous oral Zinc in the treatment of patients with carbonic anhydrase deficiency, by Benkin R. I., Martin, B. M., 1 and Agarwal, R. P.—Am J Med Sci 1999 Dec. 3; 18(6):392-405). The Carbonic Anhydrase VI produced acted as growth factors of stem cell taste buds in Patients. Thus it is shown that the synthesis/secretion of carbonic anhydrase can, indeed, be stimulated by compounds administered orally and also this has been shown that Carbonic anhydrase isozymes can be administered as growth factors of stem cells, and hence an alternative to stem cell therapy.

Carbonic anhydrase III has been used as an anti-oxidant in mice deficient in CA-III gene. In IUBMB Life 2004 June; 56(6):343-7, Anti-oxidative response of carbonic anhydrase III in skeletal muscle by Zimmerman U J, Wang P, Zhang X, Bognanovich S, Poster R

Anti-cancer Res. 1993 September-October; 13(5a):1549-56 by Teicher Ba, Liu S D, Lui J, Bolden S A, Berman T S

Acetazolamide a carbonic anhydrase inhibitor has been used as an adjuvant for the treatment of cancer for its acidifying effect together with other anti-cancer drugs to make them more cytotoxic.

### Description of Best Mode

REFFERING to FIG. 2, we observed two parallel paths of cell destruction that can be directly linked to deficiencies of cell-specific carbonic anhydrase enzymes, whether the decreased level of CA is a primary deficiency or secondary deficiency, as described therein. One path relates to the breakdown of the chemical reaction shown in Eq.1 and the other relates to the release of caspase, leading to apoptosis. The result of both paths is dead and dying cells, which include brain cells and other neural cells. Here we show that at least one cause of the destruction of brain cells and other neurons is traceable to the decreased levels of cell specific carbonic anhydrase enzymes. Heretofore, researchers had identified only one of these parallel paths, the one involving caspase, Specifically, it has been reported in the Journal of Infectious Disease Diseases, 2000 September, 182 Suppl.1:S85-92, by F. Chai, et al that the mechanism by which Zinc deficiency (equivalent to deficiency in Zinc carrying carbonic anhydrase enzyme) induces epithelial cell death involves the activation of caspase 3 as indicated in the right half of FIG. 2. The suggestion is made from this research that Zinc (i.e., CA) may suppress a step just before the activation of the caspase and a Zinc (i.e., CA) deficiency results in a failure to suppress that step.

The path illustrated on the left half of FIG. 2 shows the decreased levels of CA (i.e., Zinc-carrying enzyme) upset the rate of reversible portion of the reaction indicated in Eq. 1, above, decreasing the formation of hydrogen ion that is the fuel for the ion pump that maintains the cell wall membrane, leading to depolarization and allowing neurotoxic substances to enter the cell, causing edema and cell death.

The path illustrated in FIG. 1C is newly presented in this instant Invention. The decreased levels of Carbonic anhydrase isozymes leads to the decreased levels of hydrogen ions needed to neutralize the reactive oxygen species(ROS) produced as a result of aerobic respiration in the mitochondria of all living things and to neutralize all other reactive substances.

The path illustrated in FIG. 18 is newly presented in this instant Invention, hydrogen ions produced by cell specific carbonic anhydrase Enzymes combines with adenosine diphosphate (ADP) to produce adenosine Triphosphate (ATP) as fuel for all cellular functions (H++ADP-ATP).

Whereas in aging, there has been observed a progressive decrease in levels of enzymes of which carbonic anhydrase is one, I believed that replenishing the carbonic anhydrase enzymes that catalyze the reversible reaction of Equation 1 will at least slow the progressive and gradual death of all cells, including cells in the brain, which brain cell reduction is a major contributor to various age-related brain disorders involving dementia such as alzheimer's disease, other neurodegenerative diseases or conditions and other aged related disorders or conditions in humans.

The progressive decrease of cell specific carbonic anhydrase enzymes also results in the progressive decrease of hydrogen ions acted upon by the cytochrome system which is needed to neutralize reactive substances including but not limited to reactive oxygen species (ROS) produced during cell respiration resulting in oxidative damages to all cellular and non-cellular structures, as illustrated in FIG. 1, hence the need to replenish the cell specific carbonic anhydrase enzymes in a subject.

Specific Carbonic Anhydrase Enzymes I,II,III,IV,V,VI,VII have never been used to restore to a higher level the Carbonic anhydrase enzymes that are lacking due to decreased levels due to normal aging, diseases associated with aging which includes cancer due to oxidative stress or whether the replenishing enzymes are naturally produced and harvested or synthetically produced, nor has anyone used for this purpose any carbonic anhydrase stimulators to stimulate a patient's production of carbonic anhydrase enzymes.

As far as can be determined from the literature, one or more Cell Specific Carbonic Anhydrase enzymes have never been used as a treatment or prophylaxis for the treatment of immune diseases, whether administering one or more compounds that increases the level of one or more Cell Specific Carbonic Anhydrase enzymes that are present in decreased levels due to immune disorders of Humans, which include but are not limited to alzheimer's disease, systemic lupus erythematosus, rheumatoid arthritis, multiple sclerosis, ulcerative colitis immune diabetes mellitus, pancreatitis, colitis, syogren's disease, hepatitis, nephritis, myositis.

Likewise, No one has ever used one or a combination of one or more cell specific carbonic anhydrase enzymes I to VII PER SE in treating aging and disorders of aging in a human subject.

Likewise no one has ever used one or more compounds which naturally generates the production of one or more cell specific carbonic anhydrase enzymes I to VII as growth factors for replacing damaged or diseased cells or tissues and as growth factors of human stem cells whether they are adult or embryonic stem cells.

Nor has anyone used Carbonic anhydrase enzymes I to VII as an alternative for stem cell in the treatment of diseases or conditions.

Likewise no one has ever used a compound that increases the level of Cell specific Carbonic anhydrase I,II,III,IV,V,VI,VII as an ANTIOXIDANT in treating diseases or conditions associated with oxidative stress.

Carbonic Anhydrase isozyme inhibitors I to VII has never been used in the treatment of Cancer in humans before acetazolamide was used in the 1990″s as adjuvant treatment for lung cancer for its acidifying effect.

Experimental Oncology 22, 162-164, 2000(September) by I. Puscas, C. Puscas, M. Coltau, M. Baican, G. Domuta—The Serum of Carcinoma Patients powerfully activates Carbonic Anhydrase II.

They found out certain Cancer of the Esophagus, Colon and rectum, pancreas, lung, breast, ovary, testis, prostate activates Carbonic anhydrase II

Anti cancer Res. 1993 September-October; 13(5A):1549-56 -by Teicher B A, Liu S D, Liu J T, Holden S A, Herman T S—A Carbonic anhydrase inhibitor as a potential modulator for cancer therapies Carbonic anhydrase enzyme inhibitor acetazolamide has been used for treating cancer. They postulate that the mechanism may be involved in the ability of acetazolamide to acidify the intratumoral environment.

Here they do not emphasize the importance of taking the level of Carbonic anhydrase isozyme from the tissue (cancer) before giving the drug acetazolamide. Their teachings, suggestions and motivations are different from that of the inventor.

Eur J Med Chem. 2000 September; 35(9):876-74—by Supuran C T, Scozzafava Carbonic anhydrase inhibitors—Part 94. 1,3,4,-thiadiazole-2-Thiadazole-2-sulfonamide against CAII and CA VI as antitumor agents.

They teach that this compound is an inhibitor of Carbonic Anhydrase isozyme I and II against several leukemia, non-small cell lung cancer, ovarian, melanoma, colon, CNS, renal, prostate and breast cancer lines. The mechanism was unknown, but it might be due to inhibition of several Carbonic anhydrase isozymes (CA IX, CA XII, CA XIV) or acidification of the intracellular environment as a consequence of Carbonic anhydrase isozyme inhibition.

They do not know the mechanism by which the hydrogen ions produced by cell specific carbonic anhydrase acted upon by the cytochrome system inside the mitochomdria of all living things as in FIG. 1, FIG. 2, and FIG. 3. are used by the cells in cell metabolism.

I have come to realization that administering supplemental cell specific carbonic anhydrase enzymes or administering cell specific carbonic anhydrase stimulators or inhibitors, diseases associated with aging such as but not limited immune diseases, cancer can be prevented or treated. This can be achieved by maintaining near normal levels of cell specific carbonic anhydrase enzymes of a subject either by increasing or decreasing the levels of one or a combination of one or more cell specific carbonic anhydrase enzymes that are present in the tissue of the subject.

This method can be done by directly administering the cell specific carbonic anhydrase enzymes themselves, administering one or more compounds that stimulates the production of one or more cell specific carbonic anhydrase enzymes.

For the treatment of cancers associated with an increased expression of cell specific carbonic anhydrase enzymes in the tissues, Cell specific carbonic anhydrase inhibitors are administered in a non toxic and pharmaceutically effective dose to reduce the level of the Carbonic anhydrase isozymes back to normal levels. Maintainance dose is continued till the cancer cells goes back to normal cells.

Cell specific carbonic anhydrase enzymes I-VII can also be used as growth factors in treating diseases .This can be achieved by assaying which one or a combination of one or more cell specific carbonic anhydrase enzyme is or are present in the diseased or damaged cell or organ to be treated and then, Administering one or a combination of one or more compounds that increases the level of one or more cell specific carbonic anhydrase I-VII that are present in a specific cell or tissue to be replaced, either because of disease or from injury, then maintaining the normal levels of the same Cell Specific Carbonic anhydrase enzymes from I-VII that is present in the subject's cell or tissue to be replaced, diseased, or damaged.

This treatment can be administered to patients exhibiting diseases associated with aging such as but not limited to immune diseases, cancer and other related diseases of aging. It is also feasible to administer this treatment as a prophylactic or preventative diseases associated with aging to prevent or at least delay the onset of diseases associated with aging such as chronic neurodegenerative diseases, including but not limited to alzheimer's disease, multiple sclerosis, parkinson's disease, amyotrophic lateral sclerosis (Lou Gehrig's disease). This present treatment can be administered to prevent or at least delay age related disorders of aging which includes but not limited to the central nervous system, digestive system, excretory system, integumentary system, circulatory system, musculoskeletal system, reproductive system.

These compounds can be used as antioxidants in the prophylactic and therapeutic treatment of diseases associated with oxidative stress such as but not limited to aging, immune diseases, cancer.Oxidative stress is characterized by decreased level of hydrogen ions produced as a result of decreased levels of carbonic anhydrase isozymes, which maybe due to normal aging or conditons or diseases described in FIG. 2. This can be achieved by measuring the levels of Malondialdehyde (MDA), use of monoclonal antibodies, liquid chromography, creatine kinase before and after administering a compound that increases the level of Cell Specific Carbonic Anhydrase I,II,III,IV,V,VI,VII, other methods are available.

Thus, I disclose here a method for the treatment and prophylaxis of diseases associated with aging associated with an increase or decrease presence of cell specific carbonic anhydrase I-VII in the tissue of a patient such conditions associated but not limited to Cancer, aged related disorders, as growth factors of stem cells, as an alternative to stem cell therapy.

The compound used could be the cell specific carbonic anhydrase that is believed to be evidencing decreased presence as measured in the blood tests or in cell cultures from biopsied tissues or from the cerebro-spinal fluid. Alternatively, the compound used could be synthetically produced cell specific carbonic anhydrase enzymes. As, alternative the compound used colud be naturally produced cell specific carbonic anhydrase enzymes. Yet another alternative allows that the compound used is a compound that, when administered to a patient, promotes the natural production of the cell specific carbonic anhydrase enzyme that is evidencing decreased presence as measured in blood test or in cell cultures in biopsied tissues or from the cerebro-spinal fluid. The compound itself need not be the one that passes the blood brain barrier;the cell specific carbonic carbonic anhydrase enzyme need not be produced in the brain for it is known to pass the blood brain barrier so the promoting of natural production of cell specific carbonic anhydrase enzyme can take place elsewhere in the body.

The compound used is one or more compounds that decreases the level of one or more Cell specific carbonic anhydrase enzymes in the tissue of a patient whose levels are increased, decreasing the Carbonic anhydrase isozymes back to normal levels.

Examples of compounds that are known to decrease the presence of the required Cell specific carbonic anhydrase enzymes include but are not limited to acetazolamide and its derivatives, sulfonamides incorporating 1,2,4-triazine moieties, sulfonamide and its derivatives. derivatives of benzene sulfonamides inhibit CA II, CA V; CA IV best inhibited with phosphates, phosphonates. CA I inhibited by carbamoyl phosphate, Na Azide and Potassium cyanide are universal Carbonic anhydrase inhibitors.

Examples of compounds that are known to increase the producton of the required cell specific carbonic anhydrase enzymes include but are not limited to Zinc, growth hormones, androgens including DHEA(dehydro-epiandrosterone), sex hormones which includes estrogen, testosterone acetylcholine, melatonin, 1,25-dihydroxyvitamin D3 including vitamin D3 (cholicalciferol), NSAID (non steroidal anti-inflammatory drugs) including indomethacin; cysteamine; phorbol myristate acetate, histamine, serotonin; selective serotonin re-uptake inhibitors including sertraline, citalopram, fluoxetine. Zinc increases the production of Carbonic anhydrase VI; growth hormones increases the production of Carbonic anhydrase II, III; NSAID (non steroidal anti-inflammatory drugs) increases the production of Cell specific Carbonic anhydrase I, II; sex hormones estrogen, testosterone increases the production of carbonic anhydrase III; 1,25 dihydroxyvitamin D3 increases the production of Carbonic anhydrase II; Histamine and cysteamine increases the producion of Carbonic anhydrase I, II, IV; the selective serotonin reuptake inhibitors which includes sertraline, fluoxetine, citalopram increases the production of carbonic anhydrase I, II; acetylcholine increases the production of carbonic anhydrase serotonin and melatonin increases the production of CA-I, II,; L and D phenylalanine increases the production Of CAI, II, V(A), VII; androgens (including DHEA-dehydroepiandrosterone) increases CA-II, III production.

Administering the compound maybe done but not limited to injection or ingestion. The injection method used maybe intramuscular or intravenous, dissolved in a sterile saline solution, glucose solution, or other commonly administered parenteral solution. The best method of administering the compound will be learned with modest experimentation. The individual subject's response to the compound will be learned through testing for the cell specific enzyme in blood samples taken before and after admiministering the medication and by enzyme levels measured from cell cultures of biopsied tissues from the patient or found in the cerebro-spinal fluid. The goal is to increase or decrease the tissue level of cell specific carbonic anhydrase enzymes from an increased or decreased level in the tissue of a patient to a more normal level. Insofar as the enzyme in the blood is a reflection of the enzyme level in the tissue of a pateint, the blood test may be a sufficient indicator. In addition, other means of measuring enzyme levels that are known to the practitioner maybe employed.

For the use of cell specific carbonic anhydrase enzymes as growth factors, the main objective is to keep the cell specific carbonic at normal levels in the damaged or diseased tissue as an environment for them to differentiate and grow. This can be done by determining first, which cell specific carbonic anhydrase enzymes are present in the diseased or damaged human tissue then administering a pharmacologically effective non toxic amount of one or a combination of one or more compounds that increases the level of one or more cell specific carbonic anhydrase enzymes that is present in the damaged or diseased human tissue. Then maintaining the cell specific carbonic anhydrase enzymes at normal levels. Measuring the level of one or more cell specific carbonic anhydrase enzymes can be done directly from the damaged or diseased tissue. Other methods known can be used by the practitioner. The same procedure is followed as an alternative for stem cell therapy.

The response of the treatment can be assessed by biopsy of the tissues, imaging of the tissues involved which includes but not limited to magnetic Resonance Imaging, assessing the ameloriation of the symptoms of the disease or conditions, radioactive scannings for cancers. Other methods are available for the ordinary person skilled in the art.

Pharmaceutical composition suitable for use in the invention include compositions suitable wherein the active ingredient are contained in an effective amount to achieved the intended purpose. The determination of an effective dose is well within the capability of those skilled in the art.

A therapeutically effective dose refers to that amount of active ingredient that ameliorates the symptoms or condition, the condition being caused by or reflected in the reduced or increased concentration of cell specific carbonic anhydrase enzymes. Therapeutic efficacy and toxicity maybe determined by standard procedures from blood testing, from biopsied tissues, and by other means known to the practitioneer, for comparison with the normal values. The dosage is preferably within the range of circulating concentrations that are efficacious with little or no toxicity. The dosage varies within this range depending upon the dosage form employed, sensitivity of the patient and the route of administration.

The exact dosage will be determined by the practitioner, in light of factors related to the subject that requires treatment. Dosage and administration are adjusted to provide sufficient levels of the active moiety or to maintain the desired effect, which is a near-normal level of the cell specific carbonic anhydrase enzyme. Factors which maybe taken into account include the severity of the increased or reduction extant in the subject, general health of the subject, age, weight and gender of the subject, diet, time and frequency of administration, drug combination(s), reaction, sensitivities, and tolerance/response to therapy. Long acting pharmaceutical compositions may be administered every 3 to 4 days, every week, or once every two weeks depending on the half-life and clearance rate of the particular formulation.

Normal dosage amounts may vary from 0.1 to 100,000 micrograms, up to a total dose of about one gram, depending upon the route of administration. Guidance as to particular dosages and methods of delivery is provided in the literature and generally available to practitioners in the art. Those skilled will employ different formulations to achieve the desired results.

I claim:

1. A method for the prophylaxis and treatment of one or a combination of one or more aging conditions or disorders including cancer in a human patient comprising:
(a) Assaying for the increase or decrease level of one or a combination of one or more Cell Specific Carbonic Anhydrase CA)-I, (CA)-II, (CA)-III, (CA)-IV, (CA)-V, (CA)-VI and (CA)-VII in the tissue of a human patient suspected of having a condition of cancer which includes cancer of the breast, the colon, the prostate, the rectum, the pancreas, the lung, the ovary, the testis, the esophagus, the liver
(b) Assaying for the decreased level of one or a combination of one or more cell specific carbonic anhydrase enzymes(CA)-I, (CA)-II, (CA)-III, (CA)-IV, (CA)-V, (CA)-VI and (CA)-VII in a subject suspected of a condition of aging which includes chronic neurodegenerative disorders; immune disorders; muskulo-skeletal disorders, cardio-vascular disorders, disorders of the reproductive system, integumentary disorders; endothelial disorders; astro-intestinal disorders, genito-urinary tract disorders, amyloid disorders; myopathies
(c) selecting the human patient whose assay shows an increase in the level of one or a combination of one or more cell specific carbonic anhydrase enzymes I to VII
(d) selecting the patient whose assay shows a decrease in the level of one or a combination of one or more cell specific carbonic enzymes I to VII.
then
(e) Administering to the patient a pharmaceutically effective non-toxic amount of one or a combination of one or more Cell specific Carbonic anhydrase enzymes (CA)-I, (CA)-II, (CA)-III, (CA)-IV, (CA)-V, (CA)-VI, and (CA)-VII whose levels are decreased in the tissue of a human patient.
(f) Administering to the patient a pharmaceutically effective non toxic amount of one or a combination of one or more inhibitors of cell specific carbonic anhydrase Enzyme (CA)-I, (CA)-II, (CA)-III, (CA)-IV, (CA)-V, (CA)-VI and (CA)-VII whose levels are increased in the tissue of the human patient.

2. The method of claim 1, wherein step (a)(b) comprises determining the levels of carbonic anhydrase (CA)-I, carbonic anhydrase (CA)-II, carbonic anhydrase (CA)-III, carbonic anhydrase (CA)-IV, carbonic anhydrase (CA)-V, carbonic anhydrase (CA)-VI, carbonic anhydrase (CA)-VII, or a combination thereof in the blood of the patient.

3. The method of claim 1, wherein step (a)(b) comprises determining the levels of carbonic anhydrase (CA)-I, carbonic anhydrase (CA)-II, carbonic anhydrase (CA)-III, carbonic anhydrase (CA)-IV, carbonic carbonic anhydrase (CA)-V, carbonic anhydrase (CA)-VI, carbonic anhydrase (CA)-VII or a combination thereof in the cerebrospinal fluid of the patient.

4. The method of claim 1, wherein step (a)(b) comprises determining the levels of carbonic anhydrase (CA)-I, carbonic anhydrase (CA)-II, carbonic anhydrase (CA)-III, carbonic anhydrase (CA)-IV, carbonic anhydrase (CA)-V, carbonic anhydrase (CA)-VI, carbonic anhydrase (CA)-VII or a combination thereof in a biopsied tissue of the patient.

5. The method of claim 1, wherein said administering is by injection

6. The method of claim 5, wherein said administering is by intramuscular

7. The method of claim 5, wherein said administering is by intravenous

8. The method of claim 1, wherein said administering is by ingestion

9. The method of claim 1(f) is selected from the group that includes acetazolamide and its derivatives, sulfonamides and it's derivatives, Potassuim cyanide and its derivatives, phospates, phosponates and their derivatives, Na azide and its derivatives

10. A method for the treatment of a condition of a human patient in need of a growth factor for a stem cell or as an alternative for stem cell therapy comprising:
(a) assaying for the presence of one or a combination of one or more cell specific carbonic anhydrase I (CA)-I, cell specific carbonic anhydrase (CA)-II, cell specific carbonic anhydrase (CA)-III, cell specific carbonic anhydrase (CA)-IV, cell specific carbonic anhydrase (CA)-V, cell specific carbonic anhydrase (CA)-VI and cell specific carbonic anhydrase (CA)-VII in the tissue of the human patient in need of growth factors or as an alternative to stem cell therapy.
(b) Administering to the human patient in need of a growth factor Or as alternative to stem cell therapy one or a combination one or more cell specific carbonic anhydrase that is/are present in the tissue of the patient consisting of cell specific carbonic anhydrase (CA)-I, cell specific carbonic anhydrase (CA)-II, cell specific carbonic anhydrase (CA)-III, cell specific carbonic anhydrase (CA)-IV, carbonic anhydrase (CA)-V, cell specific carbonic anhydrase (CA)-VI, and cell specific carbonic anhydrase (CA-VII)

11. A method for reducing the signs and symptoms associated with age related bone disorders which includes osteoporosis, osteomalacia, arthritis; aged related chronic neurodegenerative disorders which includes alzheimer's disease, multiple sclerosis, amyotrophic lateral sclerosis (Lou Gehrig's disease), parkinson's disease; immune disorders which includes diabetes mellitus, systemic lupus erythematosus; collagen and integumentary systems which includes wrinkling of the skin; myopathies; cystic fibrosis; atherosclerosis, amyloid disease in a patient suspected of having signs and symptoms, the method comprising:
(a) assaying for a decrease level of one or a combination of one or more cell specific carbonic anhydrase enzymes I(CA-I), II(CA-II), CA-III(CA-III), IV (CA-IV), V(CA-V), VI(CA-VI) and VII (CA-VII) in a bodily sample from the said subject
(b) Administering to the subject a pharmaceutically effective non toxic amount of one or a combination of one or more Cell specific Carbonic Anhydrase Enzymes I(CA-I), II(CA-II), III(CA-III), IV(CA-IV), V(CA-V), VI (CA-VI) , and VII(CA-VII) that are present at decreased levels in a tissue of a subject.

12. The method of claim 1(b), wherein the condition of aging is aged related bone disorders which includes osteoporosis, osteomalacia, arthritis

13. The method of claim 1(b) wherein the condition of aging is aged related chronic neurodegenerative conditions includes alzheimer's disease, multiple sclerosis, Lou Gehrig's disease, parkinson's disease

14. The method of claim 1(b) wherein the condition of aging is aged related immune disorders which includes diabetes mellitus, systemic lupus erythematosus

15. The method of claim 1(b) wherein the condition is aged related disorders of the integumentary system and collagen which includes wrinkling of the skin

16. The method of claim 1(b) wherein the condition is aged related amyloid disorders

17. The method of claim 1(b) wherein the conditions are aged related myopathies

18. The method of claim 1(b) wherein the condition is Lou Gehrig's disease

19. The method of claim 1(a) wherein the condition is age related cancer of the colon, prostate, lung, ovary, testis, rectum, esophagus breast, pancreas, liver

20. The method of claim 1(b), wherein after treatment, the patient have reduced signs and symptoms of aged related bone disorders which include osteoporosis, osteomalacia, arthritis; chronic neurodegenerative disorders which includes alzheimer's disease, multiple sclerosis, amyotrophic lateral sclerosis (Lou Gehrig's disease, Parkinson's disease, Immune disorders which include Diabetes Mellitus, Systemic lupus erythematosus; disorders of integumentary system and collagen which includes wrinkling of the skin; myopathies; amyloidosis, atherosclerosis; cystic Fibrosis
